(19) United States
(12) Patent Application Publication
(10) Pub. No.: US 2016/0120980 A1
(43) Pub. Date: May 5, 2016

(54) REGIONAL CANCER THERAPY

(71) Applicant: Ex-Tek, LLC, Markham (CA)

(72) Inventor: Richard James Whitbourne, Markham (CA)

(21) Appl. No.: 14/529,945

(22) Filed: Oct. 31, 2014

(51) Int. Cl. A61K 47/10; A61K 31/513

(52) U.S. Cl. CPC A61K 47/10; A61K 31/513

(57) ABSTRACT

This invention relates to compositions and methods of treating cancers, and more particularly to regional delivery of cancer therapy compositions on surrounding internal tissues after surgery to remove or ablate cancer cells. The composition of the present invention can comprise an antineoplastic agent, such as, 5-Fluorouracil (“5-FU”) in an aqueous fluid, and a transcellular transport agent. An example of a composition of an embodiment of the present invention is 5-fluorouracil in an aqueous solution with polyethylene glycol and isopropanol.

### FIELD OF THE INVENTION

The invention relates to compositions and methods of treating cancers, and more particularly to delivery of cancer therapy compositions on surrounding internal tissues after surgery to remove or ablate a suspected cancerous tumor.

### BACKGROUND OF THE INVENTION

The female breast comprises many glands called lobules. Lobules produce milk that is sent to the nipple through small tubes called ducts. Many breast cancer tumors begin in the cells that line these ducts. Other tumors start in cells lining the lobules. These are known as ductal cancer and lobular cancer, respectively. In rare cases, breast cancer can start in other areas of the breast.

Breast cancer can be invasive or non-invasive. Invasive means it has spread from the milk duct or lobule to other tissues in the breast. Non-invasive means it has not yet invaded other breast tissue.

In recent years, there has been an explosion of life-saving treatment advances against breast cancer. Instead of only one or two options, today there's a plethora of treatment choices to fight the complex mix of cells in each individual cancer. The decisions—surgery, then perhaps radiation, hormonal (anti-estrogen) therapy, and/or chemotherapy—can feel overwhelming The therapies are often used in combinations, such as, surgery followed by radiation and/or chemotherapy.

Many patients with breast cancer have surgery to remove the cancer from the breast. Some of the lymph nodes under the arm are usually taken out and looked at under a microscope to see if they contain cancer cells.

Breast-conserving surgery, an operation to remove the cancer but not the breast itself, includes the following:
Lumpectomy: Surgery to remove a tumor (lump) and a small amount of normal tissue around it.
Partial mastectomy: Surgery to remove the part of the breast that has cancer and some normal tissue around it. The lining over the chest muscles below the cancer may also be removed. This procedure is also called a segmental mastectomy.
Total mastectomy: Surgery to remove the whole breast that has cancer. This procedure is also called a simple mastectomy. Some of the lymph nodes under the arm may be removed for biopsy at the same time as the breast surgery or after. This is done through a separate incision.
Modified radical mastectomy: Surgery to remove the whole breast that has cancer, many of the lymph nodes under the arm, the lining over the chest muscles, and sometimes, part of the chest wall muscles.

Chemotherapy may be given before surgery to remove the tumor. When given before surgery, chemotherapy is expected to shrink the tumor and reduce the amount of tissue that needs to be removed during surgery. Treatment given before surgery is called neo-adjuvant therapy.

Even if the doctor removes all the cancer that can be seen at the time of the surgery, some patients may be given radiation therapy, chemotherapy, or hormone therapy after surgery to eliminate any cancer cells that are left. Adjuvant therapy is treatment given after surgery, to lower the risk that cancer will come back.

Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the cerebrospinal fluid, an organ, or a body cavity such as the abdomen, the drugs mainly affect cancer cells in those areas (regional chemotherapy). The way the chemotherapy is given depends on the type and stage of the cancer being treated

Antineoplastic agents are compositions which act to inhibit or prevent the maturation and proliferation of malignant cells and the development of neoplasms, new tumors. Antineoplastic agents or drugs are cytotoxic, meaning that they kill normal cells; however, they are more damaging to the dividing cells, which included proliferating cancer cells, than to resting cells.

The chemicals and drugs known as antineoplastic agents may be divided into alkylating agents, antimetabolites, antitumor antibiotics, alkaloids and hormones/antihormones. Alkylating agents are capable of damaging the DNA of cells, thereby interfering with the process of replication; and examples are chlorambucil, mustine hydrochloride, busulfan, cyclophosphamide, ifosfamide, triethylene thiophosphamide (thiotepa), mechlorethamine, and melphalan, lomustine, semustine, streptozocin, dactinomycin.

Antimetabolites interfere with cancer cell's metabolism. Some replace essential metabolites thus removing their function, and others mimic functions thus competing with metabolites and inhibiting manufacture of protein in the cell. Examples are: 5-fluorouracil, capecitabine, cladribine, cytarabine, cytosine arabinoside, floxuridine, fludarabine, fluorouracil, mercaptopurine, methotrexate, and thioguanine.

Antitumor antibiotics isolated from microorganisms affect the function and/or synthesis of nucleic acids and examples are: bleomycin sulfate, dactinomycin, daunorubicin, doxorubicin, epirubicin, idarubicin, mitomycin, mitoxantrone, pentostatin, plicamycin, dactinomycin and streptozocin.

Alkaloids disrupt cell mitosis and cause metaphase arrest and examples are: vinblastine, vincristine, vindesine, and vinorelbine tartrate.

Hormones and antihormones, create an unfavorable environment for cancer cell growth and hormone examples are: estrogens, androgens, progestins, and corticosteroids. Antihormone examples are: aminoglutethimide, chlorotrianisene, flutamide, goserelin, leuprolide, and tamoxifen.

There are a variety of other drugs, which can be used in antineoplastic therapy, such as plant derivatives, including the podophyllotoxin derivatives etoposide and teniposide, as well as paclitaxel, a derivative of the Pacific yew tree. Platinum coordination compounds include carboplatin and cisplatin. Other agents include asparaginase, dacarbazine, hydroxyurea, the interferons, levamisole, mitotane, procarbazine, and tretinoin. The drugs used in antineoplastic therapy are highly toxic and it is difficult to deliver these agents to the site at which there is, or is suspected to be, cancer cells.

5-Fluorouracil is an anti-cancer (antineoplastic or cytotoxic) chemotherapy drug and is classified as an antimetabolite. It is used for: colon and rectal cancer; breast cancer; gastrointestinal cancers, including anal, esophageal, pancreatic and gastric; head and neck cancer; hepatoma (liver cancer); ovarian cancer; basal cell cancer of the skin and actinic keratosis (topically).

5-Fluorouracil may be administered intravenously, topically and the amount depends on many factors, including patient height and weight, general health or other health problems, and the type of cancer or condition being treated. Side effects of fluorouracil are usually reversible and go away after treatment is complete.

There are a variety of antineoplastic pharmaceuticals which are used for treatment of people and animals with cancer and as prophylactics in the prevention of the recurrence of cancer.

### SUMMARY OF THE INVENTION

In an embodiment of the present invention, there is a pharmaceutical composition comprising a pharmaceutically effective amount of one or more antineoplastic agents in solution with a transcellular transport agent, and in a further embodiment there is a pharmaceutical composition comprising a pharmaceutically effective amount of one or more antineoplastic agents in an aqueous solution with polyethylene glycol and/or isopropanol.

An embodiment of the present invention is the use of a pharmaceutical composition comprising a pharmaceutically effective amount of one or more antineoplastic agents in solution with a transcellular transport agent, through application of said composition on tissues exposed by a surgical opening to remove cancer cells, which application can be through means of spraying, painting or sponging said tissues.

An embodiment of the present invention is the method of delivering an antineoplastic agent comprising applying an aqueous composition comprising a pharmaceutically effective amount of one or more antineoplastic agents and a transcellular transport agent, on internal tissues at a site of a surgery to remove cancer cells. In this method the composition can comprise a pharmaceutically effective amount of 5-fluorouracil and polyethylene glycol and/or isopropanol.

An embodiment of the present invention is the use, during surgery to remove suspected cancerous cells, of a composition comprising a pharmaceutically effective amount of one or more antineoplastic agents and a transcellular transport agent for transcellular delivery of said one or more antineoplastic agents to cells in the vicinity of the suspected cancerous cells, by applying said composition to said cells in the vicinity of the suspected cancerous cells.

In an embodiment of the present invention, there is a pharmaceutical composition comprising a pharmaceutically effective amount of one or more antineoplastic agents in solution with a transcellular transport agent, for the treatment of cancer. The composition can be in the form of an aqueous solution with polyethylene glycol and isopropanol.

### DETAILED DESCRIPTION

Even if a surgeon removes all the cancer that can be seen at the time of the surgery, patients are often given radiation therapy, chemotherapy, or hormone therapy after surgery to kill any cancer cells that may remain, in order to lower the risk that cancer will come back. The present invention is an adjuvant therapy that is given immediately after surgery to remove cancer cells (such as a tumor or suspected cancerous tumor), through application, such as, spraying, of a composition comprising an antineoplastic agent and transcellular transport agent directly on the cells at the site where the tumor or cancer cells were just removed. In a further embodiment, the composition of the present invention could be applied before or during surgery.

The composition of the present invention comprising an antineoplastic agent also comprises a transcellular transport agent such that the antineoplastic agent is delivered farther into the tissue at the site where the tumor or cancer cells are removed, for treatment of cancer. This treatment of cancer is an adjuvant therapy that may be referred to as treatment for cancer since at the time of surgery it is not known if there is or will be any further cancer cells, or if they will all be removed by surgery. This therapy can also be referred to as a prophylactic or preventive treatment against potential cancer.

The normal course of cancer treatment is to remove a cancerous tumor or cancer cells and after the surgery to start a round of chemotherapy or radiation or hormone therapy etc. The major hurdle for any such adjuvant therapy is how to deliver the treatment to any potentially remaining cancerous cells since the treatment is harmful to normal cells. The present invention delivers an antineoplastic agent to tissue that has the potential to be cancerous or to cancer cells that have diffused into surrounding tissue and were not removed in surgery. The present invention is combined with surgery and can further also be combined with further adjuvant therapies that would normally be prescribed after surgery to remove a tumor or cancer cells.

The composition of the present invention can comprise an antineoplastic agent, such as, 5-Fluorouracil (“5-FU”) in an aqueous fluid, and a transcellular transport agent. One transcellular transport agent that can be combined with an antineoplastic agent, such as 5-FU, is the transdermal aqueous fluid from EX-TEK™ (Product Code 053110A). This transdermal agent is also effective as a transcellular transport agent. An embodiment of a composition of the present invention comprises water, isopropanol, 5-fluorouracil and polyethylene glycol.

Traditional chemotherapeutic drugs are compatible with blood, which is an aqueous composition. The composition of the present invention is also compatible with blood and tissue, and is an aqueous composition, with some non-aqueous components that are compatible with blood and tissue, such as, polyethylene glycol.

This mode of delivery of the compositions of the present invention is a regional drug delivery rather than a systemic drug administration. This mode of delivery enables an amount of drug to be delivered directly to the diseased or potentially diseased region as opposed to systemic drug administration. With this regional drug administration there are lower drug concentrations systemically. This delivery at the site of a surgical opening can be made by spraying or painting or sponging the tissue exposed in the surgical site area after the surgical procedure.

The compositions of a present invention can comprise antineoplastic agents that are currently in use for treatment of cancer or will be developed, with or without 5- Fluorouracil, in combination with a transcellular transport agent.

The present invention includes various compositions of antineoplastic agent(s) and transcellular transport agent and would include present and not yet developed antineoplastic agents and transcellular transport agents. Antineoplastic agents in the categories of alkylating agents, anti-metabolites, anti-tumor antibiotics, plant alkaloids/microtubule inhibitors and DNA linking agents can be used. Specific examples of presently available antineoplastic agents that can be used include but are not limited to: bendamustine, busulfan, carmustine, chlorambucil, cyclophosphamide, dacarbazine, ifosfamide, melphalan, procarbazine, streptozocin, temozolomide, asparaginase, capecitabine, cytarabine, 5-fluorouracil, fludarabine, gemcitabine, methotrexate, pemetrexed, raltitrexed, actinomycin D/dactinomycin, bleomycin, daunorubicin, doxorubicin, doxorubicin (pegylated liposomal), epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplatin.

### Example

On Dec. 10, 2008 an ovariohysterectomy and mastectomy were performed on a Lhasa Apso dog (born Jan. 18, 1999). From the mastectomy, a large mass was excised along with surrounding tissue. This mass was subsequently determined histologically to be an invasive mammary carcinoma. The dog was placed on a regime of Clavamox 1 ml twice daily orally, and

Metacam 5 mg dose orally once daily for one week. The dog recovered well over a period of two weeks.

However, the dog presented a second time on Jun. 2, 2010. A Mastectomy was performed on the 2^{nd} and 3^{rd} mammary complex on the left. During surgery, a regional therapy of an embodiment of a composition of the present invention was conducted. A 0.05% solution of 5-Fluorouracil (5-FU) in a transdermal aqueous fluid from EX-TEK™ (Product Code 053110A) was sprayed over the entire open tissue in the surgical site after the surgical procedure was completed before closure. This mode of delivery enables drug levels at the lesion site and in nearby surrounding tissue. 5-Fluorouracil was used because it is commonly used in breast cancer applications.

A total of three masses were identified and sent for histology. The mass from the 2^{nd} complex was histologically an invasive mammary carcinoma. The prognosis was guarded since 90% of dogs with these tumors survive for six months, while only 50% survive beyond two years. The mass on the 3^{rd} mammary complex was a benign mixed mammary adenoma. On Jun. 22, 2014 the dog was examined by a veterinarian and there were no signs of recurrence.

In this example, the composition of distilled water, isopropanol (“IPA”), 5-fluorouracil (“5-FU”) and polyethylene glycol (“PEG”) is comprised as follows:

| | |
|---|---|
| PEG 400 | 3.26 g |
| 99% IPA | 1.01 g |
| 5-FU | 0.05 g |
| Water, dist. | 5.02 g |

The mixture was heated for 10 minutes and formed a clear solution.

In a further embodiment of a composition of the present invention, the composition comprises:

| | |
|---|---|
| PEG 400 | 3.25 g |
| 5-FU | 0.06 g |
| Water, dist. | 3.00 g |

which mixture was heated for 20 minutes and formed a clear solution.

While embodiments of the invention have been described in the detailed description, the scope of the claims should not be limited by the preferred embodiments set forth in the examples, but should be given the broadest interpretation consistent with the description as a whole.

What is claimed is:

1. A pharmaceutical composition for the treatment of cancer, comprising:
a pharmaceutically effective amount of one or more antineoplastic agents in solution with a transcellular transport agent.

2. The pharmaceutical composition of claim 1, wherein the solution is an aqueous solution and the transcellular transport agent comprises polyethylene glycol.

3. The pharmaceutical composition of claim 1, wherein comprising the pharmaceutically effective amount of one or more antineoplastic agents comprises 5-fluorouracil.

4-5. (canceled)

6. A method of delivering an antineoplastic agent comprising applying an aqueous composition comprising a pharmaceutically effective amount of one or more antineoplastic agents and a transcellular transport agent, on internal tissues at a site of a surgery to remove cancer cells.

7. The method of claim 6, wherein the one or more antineoplastic agents comprises 5 fluorouracil.

8. The method of claim 7, wherein the transcellular transport agent comprises polyethylene glycol and isopropanol.

9. A method to remove suspected cancerous cells, comprising:
applying a composition comprising a pharmaceutically effective amount of one or more antineoplastic agents and a transcellular transport agent for transcellular delivery of said one or more antineoplastic agents to cells in the vicinity of the suspected cancerous cells.

10-11. (canceled)

12. The pharmaceutical composition of claim 3, wherein the transcellular transport agent comprises polyethylene glycol.

13. The pharmaceutical composition of claim 3, wherein the transcellular transport agent comprises polyethylene glycol and isopropanol.

14. The method of claim 6, wherein applying the aqueous composition comprises spraying, painting, or sponging.

15. The method of claim 7, wherein the transcellular transport agent comprises polyethylene glycol.

16. The method of claim 9, wherein the applying step is conducted during surgery to remove suspected cancer cells.

17. The method of claim 9, wherein applying comprises spraying, painting, or sponging.

18. The method of claim 9, wherein the one or more antineoplastic agents comprises 5-fluorouracil.
